(19) United States
(12) Patent Application Publication
(10) Pub. No.: US 2013/0296954 A1
(43) Pub. Date: Nov. 7, 2013

(54) SURGICAL TOOL FOR BENDING A ROD

(71) Applicants: David Skaggs, Los Angeles, CA (US); William Alan Rezach, Atoka, TN (US)

(72) Inventors: David Skaggs, Los Angeles, CA (US); William Alan Rezach, Atoka, TN (US)

(73) Assignee: WARSAW ORTHOPEDIC, INC., Warsaw, IN (US)

(21) Appl. No.: 13/462,547

(22) Filed: May 2, 2012

(51) Int. Cl. A61B 17/56

(52) U.S. Cl. 606/86 R

(57) ABSTRACT

A rod bender includes an elongated first member extending between a first end and a second end along a longitudinal axis. An elongated second member extends between a first end and a second end in a transverse orientation to the longitudinal axis. The first end of the second member is connected to the second end of the first member. The second end of the second member includes a wall that defines an inner cavity configured to receive a first implant, the wall having at least one lateral opening configured to receive a second implant.

### TECHNICAL FIELD

The present disclosure generally relates to medical devices employed during surgical applications, and more particularly, to a surgical tool that is configured to bend a vertebral rod.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion and implantable prosthetics. As part of these surgical treatments, spinal constructs such as vertebral rods are often used to reshape and provide support when spinal disorders and diseases have damaged sections of the spinal column. Rods redirect stresses away from a damaged or defective region while healing takes place to restore proper alignment and generally support the vertebral members. During surgical treatment, one or more rods may be attached via one or more bone fasteners to the exterior of two or more vertebral members. These rods often require bending during implantation in order to provide the proper shape of the spine and realignment of the vertebrae. This disclosure describes an improvement over these prior art technologies.

### SUMMARY OF THE INVENTION

Accordingly, a rod bender is provided for employment during surgical applications. It is contemplated that the rod bender is configured to bend a vertebral rod. It is also contemplated that the rod bender can be used in situ without requiring removal of the rod during a surgical procedure.

In one particular embodiment in accordance with the principles of the present disclosure, a rod bender is provided. The rod bender includes an elongated first member extending between a first end and a second end along a longitudinal axis. An elongated second member extends between a first end and a second end in a transverse orientation to the longitudinal axis. The first end of the second member is connected to the second end of the first member. The second end of the second member includes a wall that defines an inner cavity configured to receive a first implant, the wall having at least one lateral opening configured to receive a second implant.

In one embodiment, the rod bender includes a first member extending between a first end and a second end connected to a second member extending between a first end and a second end, the second member being moveable relative to the first member. The second end of the first member defines an arm having a wall that defines an inner cavity configured for disposal of a first portion of an elongated implant defining an exterior surface. The second end of the second member includes a first extension defining an inner cavity configured for disposal of a second portion of the implant and a second extension defining an inner cavity configured for disposal of a third portion of the implant. The inner cavity of the first member and the inner cavities of the first and second extensions engage the outer surface of the implant.

### BRIEF DESCRIPTION OF THE DRAWINGS

The present disclosure will become more readily apparent from the specific description accompanied by the following drawings, in which:

FIG. 1 is a perspective view of one particular embodiment of a rod bender in accordance with the principles of the present disclosure;

FIG. 2 is a perspective, close up view of one end of the rod bender shown in FIG. 1;

FIG. 2A is a perspective view of a bone fastener in accordance with the principles of the present disclosure;

FIG. 2B is a top view of the bone fastener shown in FIG. 2A;

FIG. 3 is a perspective view of one particular embodiment of a rod bender in accordance with the principles of the present disclosure wherein one end of the rod bender is partially cut away;

FIG. 4 is a perspective view of one particular embodiment of a rod bender in accordance with the principles of the present disclosure;

FIG. 5 is a perspective, close up view of a portion of the rod bender shown in

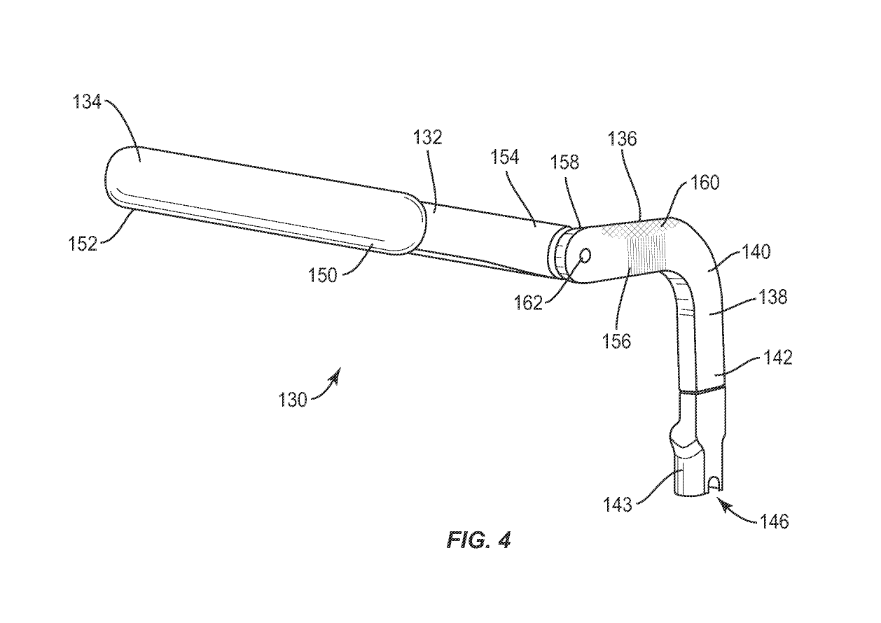

FIG. 4;

FIG. 6 is a perspective view of one particular embodiment of a rod bender in accordance with the principles of the present disclosure;

FIG. 7 is a perspective view of one particular embodiment of a rod bender in accordance with the principles of the present disclosure with parts separated;

FIG. 8 is a perspective view of one particular embodiment of a rod bender in accordance with the principles of the present disclosure engaged with a vertebral rod positioned within bone fasteners;

FIG. 9 is a perspective, close up view of one end of the rod bender shown in FIG. 8 engaged with the rod shown in FIG. 8 positioned within the bone fasteners shown in FIG. 8; and

FIG. 10 is a perspective view of the vertebral rod shown in FIG. 8.

Like reference numerals indicate similar parts throughout the figures.

### DETAILED DESCRIPTION OF THE INVENTION

The exemplary embodiments of the rod benders disclosed are discussed in terms of medical devices employed during surgical applications and more particularly, in terms of rod benders configured to bend a vertebral rod in situ. It is envisioned that the rod benders of the present disclosure may allow for single-handed operation. It is further envisioned that rod benders of the present disclosure may be configured to fit between adjacent bone fasteners to allow for easier placement in the thoracic spine by limiting the amount of space occupied by the rod benders. The rod benders of the present disclosure may therefore bend a rod coronally between two bone fasteners that are too close together to accommodate existing coronal rod benders. It is contemplated that the rod benders may be adjustable to allow a medical practitioner to have more control over the plane in which the rod is bent, such as, for example, the coronal plane.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the present disclosure may be employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may be employed with procedures for treating the lumbar, cervical, thoracic and pelvic regions of a spinal column. The present disclosure may also be used on animals, bone models and other non-living substrates, such as for training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”. Also, as used in the specification and including the appended claims, the term “tissue” includes soft tissue, ligaments, tendons, cartilage and/or bone unless specifically referred to otherwise.

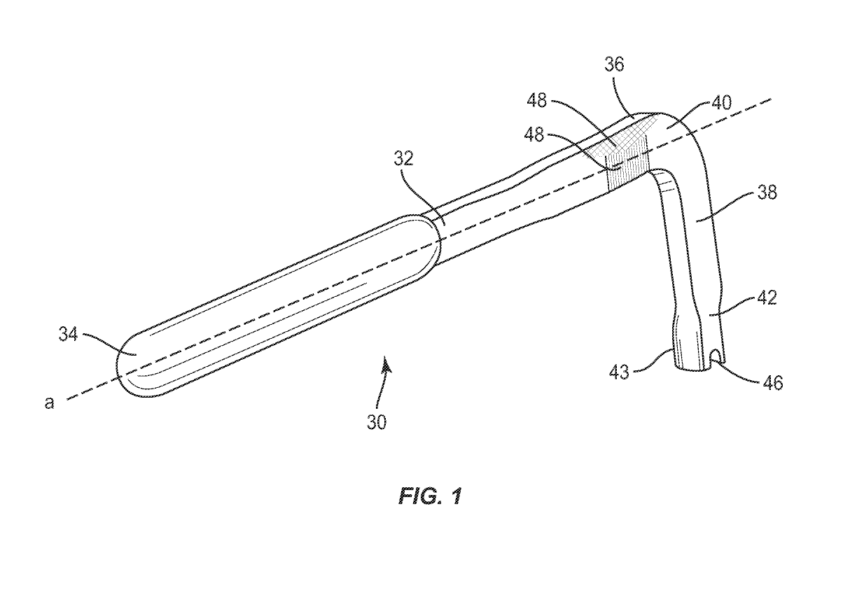

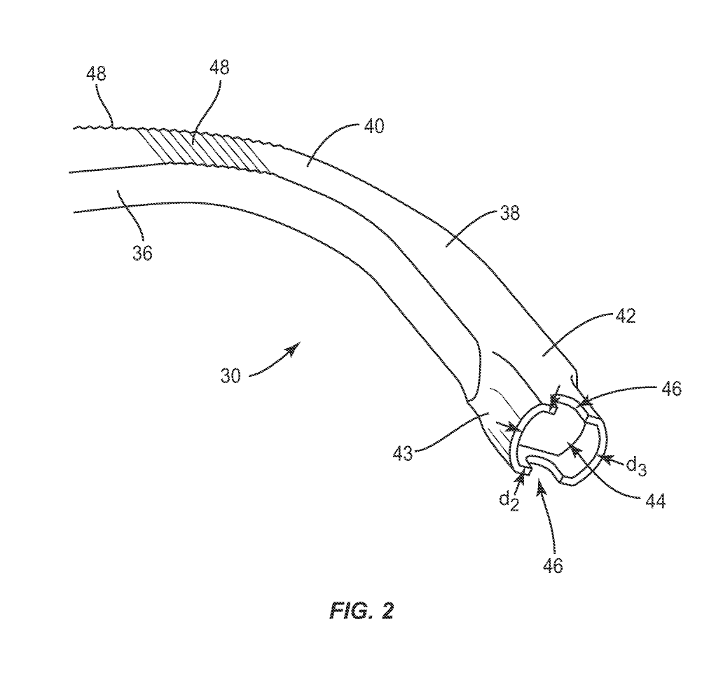

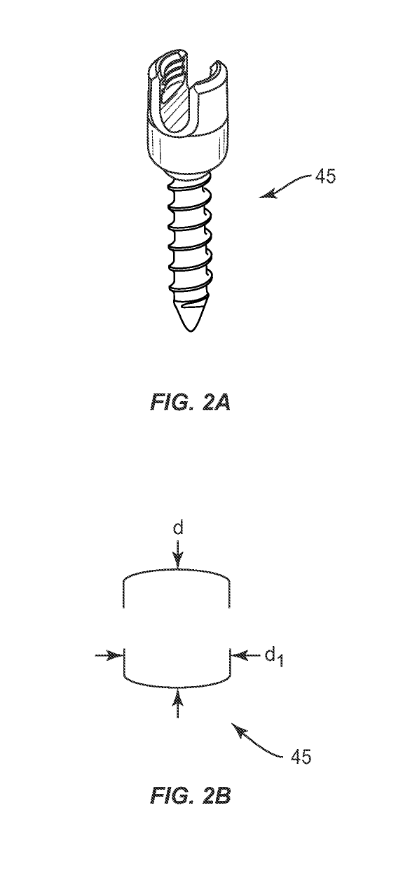

The following discussion includes a description of rod benders, related components and exemplary methods of employing the rod benders in accordance with the principles of the present disclosure. Alternate embodiments are also disclosed. Reference will now be made in detail to the exemplary embodiments of the present disclosure, which are illustrated in the accompanying figures. Turning now to FIGS. 1-2B, there is illustrated components of a rod bender 30 in accordance with the principles of the present disclosure.

Rod bender 30 is in the shape of an “L”. Rod bender 30 includes an elongated first member 32 extending between a first end, such as, for example, a proximal end 34 and a second end, such as, for example, a distal end 36 along a longitudinal axis a. An elongated second member 38 extends between a first end, such as, for example, a proximal end 40 and a second end, such as, for example, a distal end 42 in a transverse orientation to longitudinal axis a. Proximal end 40 of second member 38 is connected to distal end 36 of first member 32. In one embodiment, first and second members 32, 38 are integrally connected to one another, such that rod bender 30 is monolithic. However, it is envisioned that first and second members 32, 38 may be separate from one another and are connected via clips, a friction fit, an interference fit, pins and/or an adhesive.

Second member 38 has a length that allows proximal end 40 to be positioned outside a patient's body, while distal end is engaged with an implant implanted within the body of the patient, as will be discussed. In one embodiment, first and second members 32, 38 each have a substantially rectangular cross-section. However, the cross-sectional geometries of first and second members 32, 38 may have various configurations including, for example, round, oval, rectangular, irregular, consistent, variable, uniform and non-uniform. As shown in FIGS. 1 and 2, the upper, lower and side surfaces of first and second members 32, 38 have smooth surfaces that are not interrupted by any gaps or protrusions and are continuous between first and second members 32, 38.

Distal end 42 of second member 38 includes a wall 43 having inner and outer surfaces, the inner surface defining an inner cavity 44 configured to receive a first implant, such as, for example, a bone fastener, such as, for example, a bone fastener 45. As shown in FIGS. 2A and 2B, bone fastener 45 has arcuately shaped upper and lower surfaces separated by a distance d, extending between planar side surfaces separated by a distance d₁. In one embodiment, side surfaces of the outer surface of wall 43 are planar and are continuous with the side surfaces of second member 38. Side surfaces of the inner surface of wall 43 are planar and are separated by a distance d₂, distance d₂ being greater than distance d₁. Upper and lower surfaces of the outer surface of wall 43 are arcuately shaped. Upper and lower surfaces of the inner surface of wall 43 are arcuately shaped and are separated by a distance d3, distance d3 being greater than distance d. That is, the upper and lower surfaces of bone fastener 45 are configured to fit between the upper and lower surfaces of the inner surface of wall 43, and the side surfaces of bone fastener 45 are configured to fit between the side surfaces of the inner surface of wall 43, such that at least a portion of bone fastener 45 is received within inner cavity 44. Inner cavity 44 has a depth such that bone fastener 45 may be received securely within inner cavity 44. Inner cavity 44 is only slightly larger than the bone fastener which is received therein thereby limiting the amount of space occupied by rod bender 30 such that rod bender 30 may be engage a rod positioned within two or more bone fasteners that are close together. It is understood that that inner cavity 44 may have different dimensions depending on the geometry of the bone fastener which is to be received within inner cavity 44. That is, inner cavity 44 may be configured to receive at least a portion of any bone fastener known in the art.

Upper and lower surfaces of the exterior surface of wall 43 are tapered. That is, the width between the upper and lower surfaces of the exterior surface of wall 43 is less than the width between the side surfaces of second member 38.

Wall 43 includes at least one lateral opening 46 configured to receive a second implant, such as, for example, a vertebral rod. In one embodiment, lateral opening 46 is a pair of opposing cavities extending through side surfaces of wall 43 which define a passageway for the vertebral rod. Lateral opening 46 has an arcuate shape configured to receive at least a portion of a cylindrical vertebral rod. That is, lateral opening 46 is substantially part-cylindrical. It is envisioned that lateral opening 46 may have a radius, which is greater than the radius of the vertebral rod, which is received therein. It is further envisioned that lateral opening 46 may have a radius equal to or less than the radius of the vertebral rod, which is received therein.

In use, to bend a vertebral rod positioned into one or more bone fastener, such as, for example, bone fastener 45 anchored into the bone of a patient, such as, for example, one or more vertebra, a medical practitioner obtains access to the vertebral rod in any appropriate manner, such as through incision and retraction of tissues. It is envisioned that rod bender 30 may be used in any existing surgical method or technique including open surgery, mini-open surgery, minimally invasive surgery and percutaneous surgical implantation, whereby vertebrae is accessed through a micro-incision or sleeve that provides a protected passageway to the area. Once access to the vertebral rod is obtained, the medical practitioner positions distal end 42 of second member 38 adjacent bone fastener 45 and advances rod bender 30 distally such that at least a portion of bone fastener 45 is received within inner cavity 44. Rod bender 30 is then further advanced proximally with lateral cavity 46 aligned with the vertebral rod such that the vertebral rod is received within lateral cavity 46. After at least a portion of bone fastener 45 is received within inner cavity 44 and at least a portion of the vertebral rod is received within lateral cavity 46, the vertebral rod may be bent by manipulating first member transversely in either direction.

In one embodiment, distal end 36 of first member 32 includes at least one surface feature 48 configured to provide enhanced fixation with an instrument, such as, for example, one or more additional rod benders, such as, for example, rod bender 30, drivers, and/or rod reduction instruments. Surface features 48 on distal end 36 of first member 32 include knurling and ridges configured to create friction between rod bender 30 and a surgical instrument to resist motion between rod bender 30 and an instrument attached thereto in at least one direction and/or provide enhanced fixation with such an instrument. However, it is envisioned that surface features 48 may also include, for example, teeth, spikes, projections, indentations, roughening, or combinations thereof

In one embodiment, first member 32 includes at least one aperture extending through a side surface thereof configured to allow for the attachment of another instrument, such as, for example, one or more additional rod benders, such as, for example, rod bender 30, drivers, and/or rod reduction instruments. This feature allows rod bender 30 to incorporate one or more additional instruments such that rod bender 30 may be used in other surgical procedures. It is envisioned that rod bender 30 may be fixed to another instrument in alternative fixation configurations, such as, for example, friction fit, pressure fit, locking protrusion/recess, locking keyway and/or adhesive.

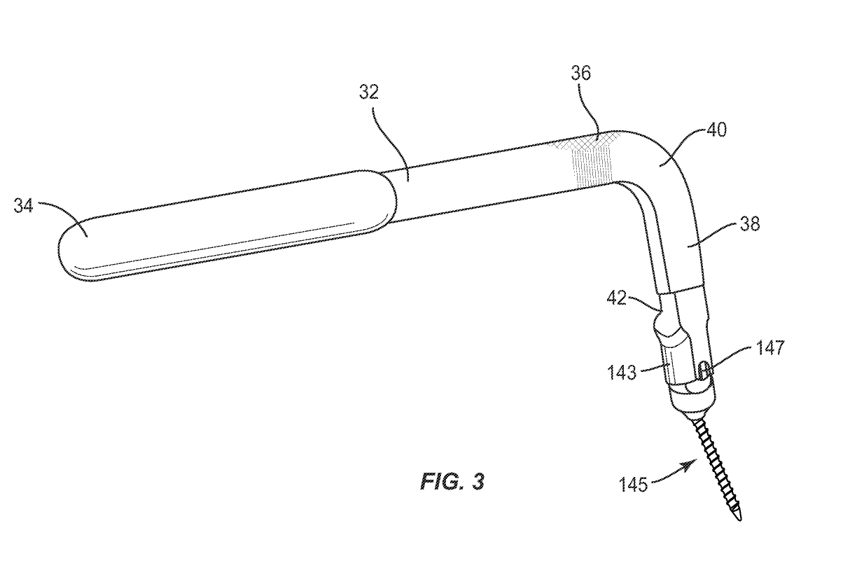

In one embodiment, shown in FIG. 3, distal end 42 of second member 38 includes a wall 143, having a configuration similar to wall 43, including inner and outer surfaces, the inner surface defining an inner cavity 144 (not shown), having a configuration similar to that of inner cavity 44 configured to receive a bone fastener 145, having a configuration similar to that of bone fastener 45, but including a break-off setscrew 147 positioned within a proximal end of bone fastener 145. Accordingly, inner cavity 144 has a depth which is greater than that of inner cavity 44, such that break-off setscrew 147 and at least a portion bone fastener 145 may be received securely therein.

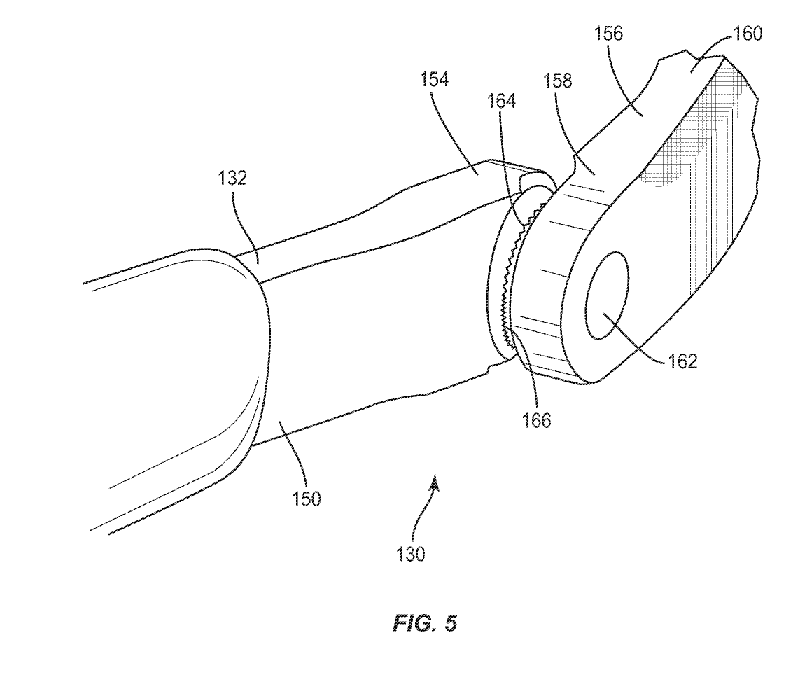

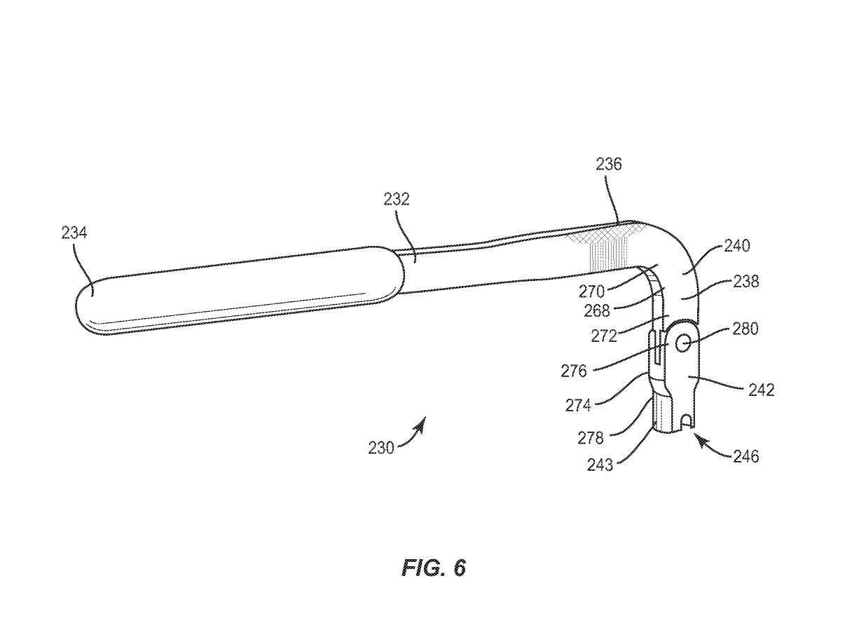

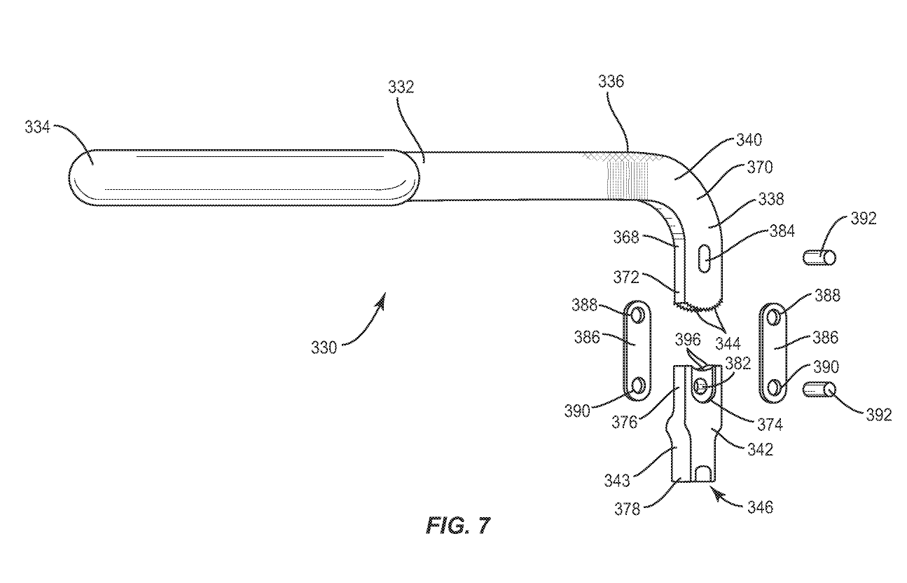

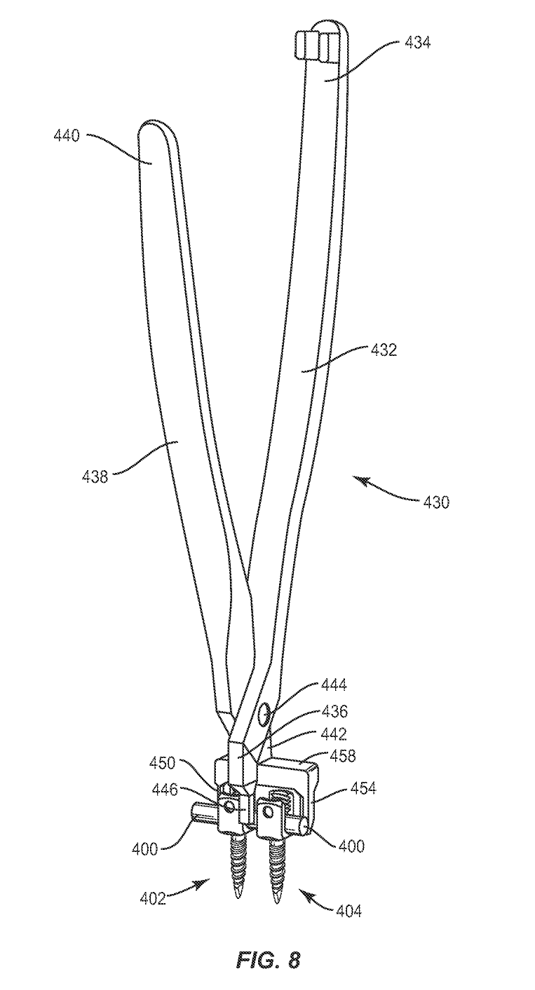

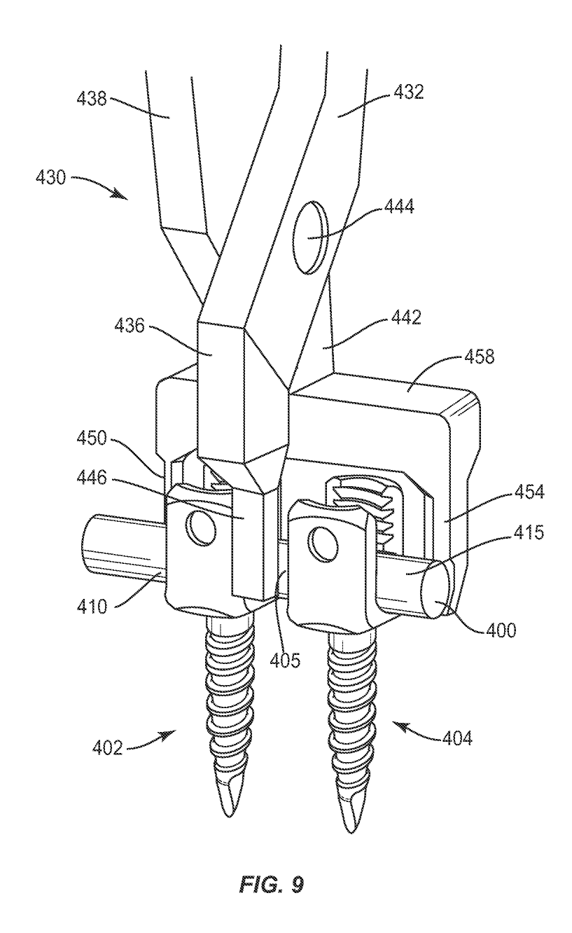

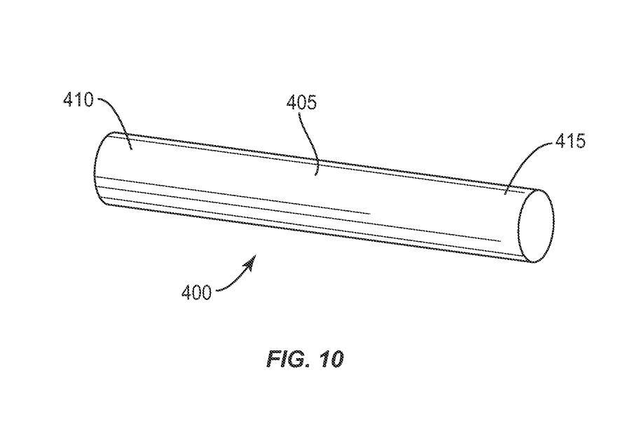

In one embodiment, shown in FIGS. 4 and 5, a rod bender 130 is provided having a configuration similar to that of rod bender 30 and includes an elongated first member 132 extending between a proximal end 134 and a distal end 136 along a longitudinal axis. An elongated second member 138 extends between a proximal end 140 and a distal end 142 in a transverse orientation to the longitudinal axis. Proximal end 140 of second member 138 is connected to distal end 136 of first member 132. Distal end 142 of second member 138 includes a wall 143 having a configuration similar to that of wall 43, described above. Wall 143 is configured to receive at least a portion of a bone fastener, such as, for example, bone fastener 45 or bone fastener 145. Wall 143 includes at least one lateral opening 146, having a configuration similar to that of lateral opening 46, described above. Lateral opening 146 is configured to receive at least a portion of a vertebral rod.

First member 132 is separated into a first portion 150 extending between a first end, such as, for example, a proximal end 152 and a second end, such as, for example, a distal end 154 and a second portion 156 extending between a first end, such as, for example, a proximal end 158, and a second end, such as, for example, a distal end 160. Proximal end 158 of second portion 156 is pivotally connected to distal end 154 of first portion 150 at a pivot joint 162 such that first portion 150 may pivot relative to second portion 156 to allow a medical practitioner to manipulate the height of proximal end 134 of first member 132 to a height that is comfortable for the medical practitioner to bend a vertebral rod by moving rod bender 30 relative to the vertebral rod. In one embodiment, first portion 150 has a length which is greater than that of second portion 156 such that pivot joint 162 is proximate to second member 138 to allow pivot joint 162 to be positioned close to the implant received within inner cavity 144 and/or the implant received within lateral cavity 146. It is envisioned that positioning pivot joint 162 in such a manner allows a medical practitioner to manipulate the height of first member 132, as discussed above, while a vertebral rod is received within lateral opening 146.

In one embodiment, proximal end 158 of second portion 156 and distal end 154 of first portion 150 include splines 164, 166 configured to mate with one another to maintain first and second portions 150, 158 in a selected position relative to one another. Splines 164, 166 include a plurality of individual spline members that extend in parallel relation about an outer surface of proximal end 158 and distal end 154. In one embodiment, splines 164, 166 are ridges or teeth configured to mesh with one another. Splines 164, 166 are configured to maintain the angular correspondence therebetween as first and second portions 150, 158 are pivoted relative to one another about pivot joint 162. That is, first and second portions 150, 158 are pivoted relative to one another about pivot joint 162 such that first portion 150 is disposed at an angle, relative to second portion 158. Splines 164, 166 maintain first and second portions 150, 158 at such an angle. In one embodiment, splines 164, 166 are spring-loaded to facilitate adjustment of first and second portions 150, 158. It is envisioned that providing such spring-loaded splines will enhance fixation between splines 164, 166 by making it more difficult for splines 164, 166 to disengage from one another. However, when a medical practitioner wishes to adjust the position of first portion 150 relative to second portion 158, for example, he or she may apply a force sufficient to overcome the force exerted by a spring of splines 164 and/or splines 166, which allows splines 164, 166 to disengage and the position of first portion 150 to be adjusted relative to that of second portion 158.

In one embodiment, shown in FIG. 6, a rod bender 230 is provided having a configuration similar to that of rod benders 30, 130 and includes an elongated first member 232 extending between a proximal end 234 and a distal end 236 along a longitudinal axis. An elongated second member 238 extends between a proximal end 240 and a distal end 242 in a transverse orientation to the longitudinal axis. Proximal end 240 of second member 238 is connected to distal end 236 of first member 232. Distal end 242 of second member 238 includes a wall 243 having a configuration similar to that of walls 43, 143, described above. Wall 243 is configured to receive at least a portion of a bone fastener, such as, for example, bone fastener 45 or bone fastener 145. Wall 243 includes at least one lateral opening 246, having a configuration similar to that of lateral openings 46, 146 described above. Lateral opening 246 is configured to receive at least a portion of a vertebral rod.

Second member 238 is separated into a first portion 268 extending between a first end, such as, for example, a proximal end 270 and a second end, such as, for example, a distal end 272 and a second portion 274 extending between a first end, such as, for example, a proximal end 276 and a second end, such as, for example, a distal end 278. Proximal end 276 of second portion 274 is pivotally connected to distal end 272 of first portion 268 at a pivot joint 280 such that first portion 268 may pivot relative to second portion 274 to allow a medical practitioner to manipulate distal end 278 of second portion 274 to position inner cavity 244 (not shown) adjacent to a bone fastener such that at least a portion of the bone fastener may be received within inner cavity 244 and/or to position lateral cavity 246 such that at least a portion of a vertebral rod is received within lateral cavity 246. Pivot joint 280 allows a medical practitioner to adjust the height of first member 232 while a vertebral rod is received within lateral opening 246 and/or move second member 238 to insert a vertebral rod in lateral opening 246 without repositioning first member 232.

In one embodiment, proximal end 276 of second portion 274 has a recess extending in a transverse orientation to lateral cavity 246 configured to receive at least a portion of distal end 272 of first portion 268. Distal end 272 of first portion 268 has an arcuate shape and is separated from a bottom portion of the recess in proximal end 276 of second portion 274 a distance when first and second portions 268, 274 are connected to one another, thereby allowing room for second portion 274 to pivot relative to first portion 268 unimpeded.

In one embodiment, shown in FIG. 7, a rod bender 330 is provided having a configuration similar to that or rod benders 30, 130, 230 and includes an elongated first member 332 extending between a proximal end 334 and a distal end 336 along a longitudinal axis. An elongated second member 338 extends between a proximal end 340 and a distal end 342 in a transverse orientation to the longitudinal axis. Proximal end 340 of second member 338 is connected to distal end 336 of first member 332. Distal end 342 of second member 338 includes a wall 343 having a configuration similar to that of walls 43, 143, 243 described above. Wall 343 is configured to receive at least a portion of a bone fastener, such as, for example, bone fastener 45 or bone fastener 145. Wall 343 includes at least one lateral opening 346, having a configuration similar to that of lateral openings 46, 146, 246 described above. Lateral opening 346 is configured to receive at least a portion of a vertebral rod.

Second member 338 is separated into a first portion 368 extending between a first end, such as, for example, a proximal end 370 and a second end, such as, for example, a distal end 372 and a second portion 374 extending between a first end, such as, for example, a proximal end 376 and a second end, such as, for example, a distal end 378. Distal end 372 of first portion 368 has an arcuate shape and proximal end 376 of second portion 374 has a concave shape corresponding to the arcuate shape of distal end 372 of first portion 368, such that distal end 372 and proximal end 376 are movable relative to one another along a curvature defined by distal end 372 and/or proximal end 376 to align openings 382, 384 extending through proximal end 376 and distal end 372, respectively. Opening 382 has a circular configuration, while opening 384 has an oblong configuration such that first portion 368 can move relative to second portion 374 when a pin or a dowel is inserted into openings 382, 384 to connect first and second portions 368, 374. However, it is envisioned that openings 382, 384 may have various cross sectional configurations, such as, for example, round, oval, oblong, square, rectangular, polygonal, irregular, uniform, non-uniform, offset, staggered, tapered, consistent or variable, depending upon the particular application.

Proximal end 376 of second portion 374 and distal end 372 of first portion 368 are connected via at least one plate 386. Plate 386 has a length that is at least the distance between openings 382, 384 when first and second portions 368, 374 are connected to one another and includes a first aperture 388 in a proximal end and a second aperture 390 in a distal end thereof. Apertures 388, 390 are aligned with openings 382, 384 when first and second portions 368, 374 are connected to one another. Plate 386 is maintained in position by inserting a first dowel, such as, for example, pin 392 through aperture 388 and opening 384; a second dowel, such as, for example, pin 392 is inserted through aperture 390 and opening 382. As shown in FIG. 7, two plates 386 are utilized to connect first and second portions 368, 374. The first plate 386 may connect first and second portions 368, 374 as described above. Next, the second plate 386 is positioned such that apertures 388, 390 of the second plate 386 are aligned with openings 382, 384 on first and second portions 368, 374 on a side surface of first and second portions 368, 374 opposite the side surface in which the first plate 386 is positioned. The first dowel may then be advanced into aperture 388 of the second plate 386 and the second dowel may be advanced into aperture 390 of the second plate 386.

In one embodiment, distal end 372 of first portion 368 may have friction elements, such as, for example, teeth configured to engage cavities 396 in proximal end 376 of second portion 374 to maintain the angular correspondence between first and second portions 368, 374 such that openings 382, 384 are aligned with one another such that a pin or dowel may be inserted through openings 382, 384 to connect first and second portions 368, 374.

A kit for bending a rod is also contemplated. The kit for bending a rod includes a rod bender 330 as described herein. The kit also includes one or more second portions 374 attachable to first portion 368 each having walls 343 having inner cavities 344 configured to receive bone fasteners having different sizes and shapes. It is contemplated that the kit can include second portions 374 configured to receive bone fasteners having different sizes and shapes as well as different types of bone fasteners, in addition to bone fasteners 45, 145 described herein.

In one embodiment, shown in FIGS. 8-10, a rod bender 430 is pliers style instrument including a first member 432 extending between a first end, such as, for example, a proximal end 434 and a second end, such as, for example, a distal end 436 connected to a second member 438 extending between a first end, such as, for example, a proximal end 440 and a second end, such as, for example, a distal end 442. First and second members 432, 438 are connected to one another at a pivot joint 444 such that second member 438 is moveable relative to first member 432. Proximal end 434 of first member 432 defines an elongated handle portion configured for gripping by a medical practitioner.

An inner surface of first member 432 defines a channel configured to receive at least a portion of second member 438 when first and second members 432, 438 are moved relative to one another such that proximal ends 434, 440 converge. First member 432 has a reduced width distal to the channel. Pivot joint 444 extends through the portion of first member 432 having a reduced width distal to the channel. Distal end 436 of first member 432 has a width which is approximately the same as the portion of first member 432 having a reduced width distal to the channel. An inner surface of second member 438 defines a channel configured to receive at least a portion of first member 432 when first and second members 432, 438 are moved relative to one another such that proximal ends 434, 440 converge. Second member 438 has a reduced width distal to the channel. Pivot joint 444 extends through the portion of second member 438 having a reduced width distal to the channel. Distal end 442 of second member 438 has a width which is approximately the same as the portion of second member 438 having a reduced width distal to the channel.

Distal end 436 of first member 432 defines an arm 446 having a wall that defines an inner cavity 448 (not shown) configured for disposal of a first portion, such as, for example, a first portion 405 of an elongated implant, such as, for example, vertebral rod 400. Arm 446 has a width which is approximately the same as distal end 436. Distal end 442 of second member 438 includes a transverse bridge 458 extending between a first a first end, such as for example, an anterior end and a second end, such as, for example, a posterior end. The anterior end of transverse bridge 458 has a first extension 450 extending distally therefrom defining an inner cavity 452 (not shown) configured for disposal of a second portion of vertebral rod 400, such as, for example, a second portion 410. The posterior end of transverse bridge 458 has a second extension 454 extending distally therefrom defining an inner cavity 456 (not shown) configured for disposal of a third portion of vertebral rod 400, such as, for example, a third portion 415. First and second extensions 450, 454 each have a width which is approximately the same as the width of arm 446.

Inner cavity 448 of first member 432 extends in a direction substantially orthogonal to arm 446. Inner cavities 452, 456 of second member 438 extend in a direction substantially orthogonal to first and second extensions 450, 454. Inner cavity 448 of first member 432 and inner cavities 452, 456 of first and second extensions 450, 454 engage the outer surface of vertebral rod 400. As such, inner cavity 448 of first member 432 and inner cavities 452, 456 of first and second extensions 450, 454 are linear. That is, inner cavities 448, 452, 456 are substantially coaxial.

In one embodiment, inner cavities 448, 452, 456 are substantially part-cylindrical, such that inner cavities 448, 452, 456 may engage a vertebral rod which is cylindrical, such as, for example, vertebral rod 400. It is envisioned that inner cavities 448, 452, 456 may each have a radius which is greater than the radius of vertebral rod 400. It is also envisioned that inner cavities 448, 452, 456 may each have a radius which is equal to or less than the radius of vertebral rod 400.

Rod bender 430 may engage vertebral rod 400 when vertebral rod 400 is positioned within at least two bone fasteners, such as, for example, bone fasteners 402, 404 when bone fasteners 402, 404 are anchored into at least one bone of a patient, such as, for example, one or more vertebra. As shown in FIGS. 8 and 9, rod bender 430 may be manipulated such that arm 446 is positioned between bone fasteners 402, 404 such that at least a portion of first portion 405 of vertebral rod 400 is received within inner cavity 448 of arm 446. At the same time, at least a portion of second portion 410 of vertebral rod 400 is received within inner cavity 452 of first extension 450 and at least a portion of third portion 415 of vertebral rod 400 is received within inner cavity 456 of second extension 454. As such, rod bender 430 engages vertebral rod 400 in three locations along the length of vertebral rod 400, the three locations being spaced apart from one another. A force may be applied to converge proximal ends 434, 440 of first and second members 432, 438. As proximal ends 434, 440 converge, distal ends 436, 442 of first and second members 432, 438 converge as well, causing vertebral rod 400 to bend at first portion 405. That is, at least a portion of second and third portions 410, 415 of vertebral rod 400 are positioned within inner cavities 452, 456 to maintain vertebral rod 400 in position as arm 446, with at least a portion of first portion 405 of vertebral rod 400 positioned within inner cavity 448, applies force to first portion 405 to bend vertebral rod 400 at first portion 405.

According to one embodiment, the rod benders of the present disclosure are made of an autoclavable material. That is, a material capable of withstanding high temperatures and pressures used to sterilize surgical materials. As such, the rod benders of the present disclosure may be fabricated from a metal, metal alloy, polymer, ceramic material, or combinations thereof. A suitable metal and/or metal alloy can include transition metals, iron (e.g., steel), titanium, nickel, chromium, cobalt, cobalt chrome (CoCr), tungsten, molybdenum, vanadium, and any combination thereof.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical tool for bending a rod comprising:
an elongated first member extending between a first end and a second end along a longitudinal axis;
an elongated second member extending between a first end and a second end in a transverse orientation to the longitudinal axis;
wherein the first end of the second member is connected to the second end of the first member; and
wherein the second end of the second member includes a wall that defines an inner cavity configured to receive a first implant, the wall having at least one lateral opening configured to receive a second implant.

2. The surgical tool of claim 1, wherein the at least one lateral opening is a pair of opposing cavities.

3. The surgical tool of claim 1, wherein the at least one lateral opening has an arcuate shape.

4. The surgical tool of claim 1, wherein the first implant is a bone fastener and the second implant is a vertebral rod.

5. The surgical tool of claim 1, wherein the first member is separated into a first portion extending between a first end and a second end and a second portion extending between a first end and a second end, the first end of the second portion being pivotally connected to second end of the first portion.

6. The surgical tool of claim 5, wherein the first end of the second portion and the second end of the first portion each include splines configured to mate with one another to maintain first and second portions in position relative to one another.

7. The surgical tool of claim 6, wherein at least one of the splines on the first end of the second portion and the second end of the first portion are spring-loaded.

8. The surgical tool of claim 1, wherein the second member is separated into a first portion extending between a first end and a second end and a second portion extending between a first end and a second end, the first end of the second portion being connected to the second end of the first portion.

9. The surgical tool of claim 8, wherein the first end of the second portion and the second end of the first portion are connected via at least one plate.

10. The surgical tool of claim 8, wherein the first end of the second portion is pivotally connected to the second end of the first portion via a pin extending through the first end of the second portion and the second end of the first portion.

11. The surgical tool of claim 1, wherein the second end of the first member includes at least one surface feature selected from the group consisting of teeth, spikes, ridges, projections, indentations, roughening, knurling, or combinations thereof

12. A kit comprising:
the tool of claim 8; and
a plurality of second portions having inner cavities configured to receive first implants having different sizes and shapes.

13. A surgical tool for bending a rod comprising:
a first member extending between a first end and a second end connected to a second member extending between a first end and a second end, the second member being moveable relative to the first member;
wherein the second end of the first member defines an arm having a wall that defines an inner cavity configured for disposal of a first portion of an elongated implant defining an exterior surface;
wherein the second end of the second member includes a first extension defining an inner cavity configured for disposal of a second portion of the implant and a second extension defining an inner cavity configured for disposal of a third portion of the implant; and
wherein the inner cavity of the first member and the inner cavities of the first and second extensions engage the outer surface of the implant.

14. The surgical tool of claim 13, wherein the first, second and third portions of the implant are spaced apart from one another.

15. The surgical tool of claim 14, wherein the first portion of the implant is positioned between the second and third portions thereof

16. The surgical tool of claim 13, wherein the inner cavities of the first and second extensions are linear.

17. The surgical tool of claim 13, wherein the inner cavity of the first member and the inner cavities of the first and second extensions are substantially coaxial.

18. The surgical tool of claim 16, wherein the inner cavity of the first member extends in a direction substantially orthogonal to the second end of the first member and the inner cavities of the first and second extensions extend in a direction substantially orthogonal to the second end of the second member.

19. The surgical tool of claim 13, wherein the implant is a vertebral rod.

20. The surgical tool of claim 19, wherein the inner cavity of the first member and the inner cavities of the first and second extensions are substantially part-cylindrical.
